(19) United States
(12) Patent Application Publication
(10) Pub. No.: US 2026/0283999 A1
(43) Pub. Date: Sep. 24, 2026

(54) STAT3 INHIBITOR TO TREAT SUBARACHNOID HEMORRHAGE

(71) Applicant: UNIVERSITY OF FLORIDA RESEARCH FOUNDATION, INCORPORATED, Gainesville, FL (US)

(72) Inventors: Brian Lim HOH, Gainesville, FL (US); Brandon Lucke-Wold, Gainesville, FL (US); Chenglong LI, Gainesville, FL (US)

(21) Appl. No.: 18/873,831

(22) PCT Filed: Jun. 23, 2023
(86) PCT No.: PCT/US2023/026076

§ 371 Date: Dec. 11, 2024

Related U.S. Application Data
(60) Provisional application No. 63/354,902, filed on Jun. 23, 2022.

(51) Int. Cl. A61K 31/325; A61P 7/04

(52) U.S. Cl. CPC A61K 31/325; A61P 7/04

(57) ABSTRACT

Disclosed is a method for treating subarachnoid hemorrhage (SAH) and/or reducing the incidence of subsequent cerebral vasospasm in a patient. The method involves administering a therapeutically effective amount of a first compound or a pharmaceutical composition comprising said first compound to the patient, wherein the first compound comprises a STAT inhibitor compound.

### BACKGROUND

Vasospasm and delayed cerebral ischemia (DCI) are complications of subarachnoid hemorrhage (SAH). 70% of patients with subarachnoid hemorrhage later develop cerebral vasospasm during the course of hospital stay and treatment. Only 30% have symptomatic vasospasm. It is unknown why vasospasm occurs following subarachnoid hemorrhage, or the cause of the delay in presentation of cerebral vasospasm. Moreover, methods of prevention of cerebral vasospasm are needed. Interleukin 6 (IL-6) has been identified as a key component of vasospasm development and subsequent DCI. IL-6 has been identified as a mediator of increased inflammatory cascade in vasospasm development and resulting DCI.

### SUMMARY

It has been identified that potentially targeting IL-6 directly prior to initiation of the inflammatory cascade can reduce development of vasospasm. Consequently, therapeutics targeting this pathway and the inhibition of the IL-6 inflammatory response are beneficial in treating subarachnoid hemorrhage and DCI. While the correlation of neuroinflammation and the development of cerebral vasospasm following subarachnoid hemorrhage has been studied, the mechanisms by which this occurs have not be fully fleshed out.

In certain embodiments, provided are formulations including STAT inhibitor compounds. In some aspects, the STAT includes STAT3. Disclosed are pharmaceutical compositions comprising inhibitors of STAT. STAT inhibitor compounds are also described in WO 2019/067696, which is incorporated herein by reference. In various aspects, the inhibitors of STAT can be used in methods of treating, eliminating, or preventing the inflammatory surge following subarachnoid hemorrhage and subsequent cerebral vasospasm.

In accordance with an aspect of the present disclosure, there is disclosed a method for treating subarachnoid hemorrhage (SAH) and/or reducing the incidence of subsequent cerebral vasospasm in a patient, comprising administration of a therapeutically effective amount of a first compound or a pharmaceutical composition comprising said first compound to the patient, wherein the first compound comprises a STAT inhibitor compound. In a further embodiment, the STAT inhibitor compound comprises a STAT3 inhibitor.

### BRIEF DESCRIPTION OF DRAWINGS

FIG. 1 shows a series of graphs relating to a modified garcia and turn test. p-Stat 3 inhibition improved behavior measured by modified garcia scoring and right turn test 1, 2, and 3 days following subarachnoid hemorrhage.

FIG. 2 shows a series of images and a graph showing that p-Stat 3 inhibition prevented vasospasm on day 5 following subarachnoid hemorrhage.

FIG. 3 shows a series of images and a graph showing that p-Stat 3 inhibition improved cerebral blood flow and prevented spreading cortical depressions following subarachnoid hemorrhage.

FIG. 4 Is a diagram showing how the IL-6 cascade activates the p-STAT3 pathway to induce neuroinflammation.

### DETAILED DESCRIPTION

### 1. Definitions

The term “carrier” refers to a diluent, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

By “pharmaceutically acceptable” is meant that the carrier, diluent or excipient must be compatible with the other ingredients of the formulation and not deleterious to the recipient thereof.

For the purpose of this disclosure, the term “STAT” and “signal transducer and activator of transcription” can be used interchangeably, and can refer to a protein family comprising at least the following members: STAT1, 2, 3, 4, 5a, 5b, and 6. The STAT family of proteins are latent cytoplasmic transcription factors that mediate cellular responses to cytokines, growth factors and other polypeptide ligands.

As used herein, the term “STAT3” and “signal transducer and activator of transcription 3” can be used interchangeably and refer to a transcription factor encoded by a gene designated in human as the STAT3 gene, which has a human gene map locus of 17q21 and described by Entrez Gene cytogenetic band: 17q21.31; Ensembl cytogenetic band: 17q21.2; and, HGNC cytogenetic band: 17q21. The term STAT3 refers to a human protein that has 770 amino acids and has a molecular weight of about 88,068 Da. The term is inclusive of splice isoforms or variants, and also inclusive of that protein referred to by such alternative designations as: APRF, MGC16063, Acute-phase response factor, DNA-binding protein APRF, HIES as used by those skilled in the art to that protein encoded by human gene STAT3. The term is also inclusive of the non-human ortholog or homolog thereof.

As use herein, the term “STAT inhibitor” or “STAT3 inhibitor” refers to agents that block or reduce expression or activity of STAT or STAT3, respectively. STAT or STAT3 inhibitors include the examples provided herein, or analogs, derivatives or pharmaceutically acceptable salts thereof. The STAT inhibitors can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The term “treating” or “treatment of” as used herein refers to providing any type of medical management to a subject. Treating includes, but is not limited to, administering a composition comprising one or more active agents to a subject using any known method for purposes such as curing, reversing, alleviating, reducing the severity of, inhibiting the progression of, or reducing the likelihood of a disease, disorder, or condition or one or more symptoms or manifestations of a disease, disorder or condition. In some instances as described herein, “treating” may involve reducing the effects or symptoms of SAH or cerebral vasospasm, or preventing those effects or symptoms altogether. In some embodiments, preventing cerebral vasospasm may include treating SAH in a patient.

A “therapeutically effective amount” refers to an amount which, when administered in a proper dosing regimen, is sufficient to reduce or ameliorate the severity, duration, or progression of the disorder being treated (e.g., subarachnoid hemorrhage or vasospasm), prevent the advancement of the disorder being treated (e.g., subarachnoid hemorrhage or vasospasm), cause the regression of the disorder being treated (e.g., subarachnoid hemorrhage or vasospasm), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

A “patient” or “subject” as used herein interchangeably, refers to an animal, preferably a mammal such as a non-primate and a primate and most preferably a human, wherein said human may exhibit any of the aforementioned SAH symptoms described herein. The patient may have suffered a trauma involving a head injury or aneurysm. The patient may be experiencing thrombocytopenia or other known symptoms of SAH. A suitable subject for the embodiments described herein may be a human that is suspected of having, has been diagnosed as having, or is at risk of developing cerebral vasospasm following subarachnoid hemorrhage. A suitable subject for the embodiments described herein may further be a human that is suspected of having or has been diagnosed as having SAH.

As used herein, the term “stroke” or “cerebrovascular accident” refers to the local and acute neurological symptoms and signs usually resulting from a vessel-associated disease. Strokes are either closed type (due to clogging of blood vessels) or hemorrhagic type (due to hemorrhage from blood vessels). As used herein, the term “ischemia” refers to the inadequacy of blood supply. If ischemia is severe enough and long-term neurons or other cellular component death, this condition is referred to as “infarction”.

Hemorrhage arises from the rupture of an aneurysm in the subarachnoid space (extraparenchymal), for example, in the Willis circle, resulting in subarachnoid hemorrhage (SAH). Bleeding can also be intraparenchymal hemorrhage due to, for example, rupture of a vessel damaged by long-standing hypertension, and may result in intracerebral hematomas (cerebral hemorrhage). Bleeding can be accompanied by ischemia or infarction resulting from vasospasm. The mass effect of intracerebral hematoma may impair blood supply to adjacent brain tissue. Subarachnoid hemorrhage can cause reactive vasospasm of the brain surface vessels, resulting in additional ischemic brain injury in a delayed fashion. Infarcted tissue may also be secondary to the hemorrhage. An aneurysm sometimes ruptures in the brain, causing intracerebral hematoma, and rupture into the ventricle, causing intraventricular hemorrhage.

Although most of the occlusive strokes are caused by atherosclerosis and thrombosis, and most hemorrhagic strokes are associated with hypertension or aneurysms, this type of stroke is associated with heart disease, trauma, infections, neoplasms, blood diseases dyscrasia, vascular malformations, immune disorders, and exotoxins.

### Subarachnoid Hemorrhage (SAH)

The brain is surrounded by three layers of membranes or meninges: the pia mater, the arachnoid mater, and the dura mater. The lowering of the trabecular membrane is the area between the telencephalic membrane and the arachnoid membrane surrounding the brain. The term subarachnoid hemorrhage (herein referred to as a “SAH”) refers to hemorrhage into the descending subarachnoid space. SAH can occur spontaneously from the cerebral aneurysm or can be formed from the trauma.

Symptoms include severe headaches (sometimes referred to as lightheaded headaches) that indicate rapid symptoms, vomiting, and altered levels of consciousness. Diagnosis usually consists of computed tomography (CT scanning), or often a lumbar puncture. Treatment consists of close observation, medication and early neurosurgical investigation, and treatment to prevent recurrence and complications.

SAH is a medical emergency and can even lead to death or serious disability if it is not recognized and treated at an early stage. Half of all SAH cases are fatal, and 10-15% die before reaching the hospital. SAH, considered a form of stroke, results in 1 to 7% of all strokes. When caused by rupture of an intracranial aneurysm, bleeding is seen in the subarachnoid space. Bleeding due to SAH can lead to brain damage, brain displacement, decreased cerebral perfusion, and hydrocephalus. The incidence of SAH from an intracranial aneurysm ruptured in the United States is estimated to be 1 per 10,000, with approximately 27,000 to 30,000 new SAH cases occurring annually. These ruptured aneurysms have a 30-day mortality rate of 45%. It is also estimated that 30% of survivors have moderate to severe disabilities.

Patients who survive from SAH also have the risk of secondary complications. Among these complications, the most notable are aneurysmal re-bleeding and cerebral vasospasm. Although cerebral vasospasm is a consequence of SAH, it can also occur after any disease that deposits blood at the lower epidural space. More specifically, the term cerebral vasospasm refers to the narrowing of a large cerebral artery at the base of the brain after hemorrhage into the subarachnoid space that ultimately reduces perfusion of the distal brain region.

### Symptoms of SAH

A typical symptom of SAH is Thrombocytopenia (described as a headache developed over a few seconds to a few minutes), but these symptoms only account for about ⅓ of all SAH patients. Approximately 10% of patients in need of medical care with these symptoms have a potential SAH. Other symptoms may include vomiting, and one out of 14 incidents of SAH causes seizures. Other signs including neck stiffness and meningism may be present, such as consciousness impairment, reduced levels of consciousness, or coma. Intraocular hemorrhage may occur in response to elevated pressure around the brain. The vitreous body (surrounded by a hyaloid membrane) and vitreous hemorrhage can be identified by fundoscopy. It is known as Terson syndrome (occurring in 3-13% of cases) and is more common in more severe SAHs. In patients with Thrombocytopenia, any of the signs mentioned do not help to identify or exclude bleeding, but seizures are more common if the bleeding is the result of a ruptured aneurysm contrary to other causes. Ocular motor neuropathy (affecting eye upwards and downwards, except for normal pupil reflexes and incapability of lifting the eyelids on the same side) is indicative of bleeding from an aneurysm near the posterior communicating artery. The isolated pupil dilatation may also be indicative of brain herniation because of intracranial pressure increase. The body releases large amounts of adrenaline and similar hormones because of the bleeding, which results in a sharp rise in blood pressure. The heart is put into substantial tension, and neurogenic pulmonary edema, cardiac arrhythmia, changes in electrocardiograms (sometimes markedly reversing the “cerebral” T wave), and heart attack (3%) can occur rapidly after the onset of bleeding.

SAH can also occur in people with head injury. Symptoms may include headaches, decreased levels of consciousness, or hemiparesis. This is a serious complication of head injury, particularly when associated with a low Glasgow Coma Scale level.

Diagnosis of SAH The initial step for evaluating a person suspected of having SAH is to obtain a medical history and perform physical tests. Other possible causes, such as meningitis, migraine, and cerebral sinus thrombosis, are considered at the same time, since only 10-25% of the onset patients with Thrombocytopenia suffer from SAH. Occasionally, the appearance in the brain, which is twice the SAH, is misdiagnosed as SAH.

The diagnosis of SAH cannot be made on a medical basis alone. Generally, the medical imaging of the brain (usually a CT scan with high sensitivity (especially on the first day after bleeding initiation>95% correct confirmation) is required to confirm or exclude bleeding. Magnetic resonance imaging (MRI scans) can be more sensitive after a few days compared to CT. In a person with a normal CT or MRI scan, a lumbar puncture that removes cerebrospinal fluid (CSF) from the lumbar sac to the needle shows evidence of hemorrhage in 3% of the groups found to be normal in CT; Thus, the lumbar puncture is considered mandatory if the imaging is negative. CSF samples are tested for the appearance of centrifuged fluids, xanthochromia, or by spectrophotometry for bilirubin, the breakdown product of hemoglobin in CSF.

After SAH is identified, its origin should be determined. CT angiography to visualize an aneurysm (which visualizes blood vessels as radiocontrast on a CT scan) is generally the first step, but invasive catheter angiography (a technique in which a carotid angiogram is injected through a catheter developed for the brain artery) Is a gold standard test, but has a high risk of complications. The latter is useful if you plan to remove the cause of bleeding, such as an aneurysm at the same time.

### Causes of SAH

Spontaneous SAH is most commonly caused by rupture of the aneurysm of the cerebral artery (85%). The cerebral aneurysm is weak at the walls of the enlarged brain arteries. These tend to be located in the Willis circle and its branches. Most cases of SAH are due to bleeding from the small aneurysm, but larger aneurysms (more rarely) are likely to rupture. No aneurysms are detected from the first angiogram in 15-20% of cases of spontaneous SAH. Non-aneurysmal hemorrhage in which blood is confined to the area of the midbrain leads to 10% of SAH cases. Of these, no aneurysms are generally identified. The remaining 5% cases are due to vasculitis injuries to the arteries, other diseases affecting the blood vessels, diseases of the spinal cord blood vessels, and bleeding into various tumors. Most trauma SAH occurs near the skull fracture or intracerebral contusion.

### Vasospasm

Vasospasm is the most common cause of focal ischemia after SAH. This results in up to 23% of SAH-related disorders and deaths, thus adversely affecting outcomes in patients with SAH. Of all types of ischemic stroke, vasospasm is the only one that is somewhat predictable, preventable, and treatable (Macdonald, R. L. and Weir, B. In Cerebral Vasospasm. 2001. Academic Press, Burlington, MA, USA].

Every year, about one out of 10,000 patients have ruptured aneurysms. Mortality and morbidity increase with the amount of bleeding, reflect the age and health of the patient, and the risk of developing aneurysms increases with age. Rebleeding is exceptionally disadvantageous due to an increase in the amount of SAH as well as an increased likelihood of expansion into the brain and ventricles. Most deaths from rupture of an aneurysm occur within a short time after discharge or due to the effects of early bleeding or premature rebleeding. Possible signs and symptoms from vasospasm typically occur only in patients who survive the first few days. These signs and symptoms can be used to identify the “patient” or “subject” to be treated according to the methods described herein.

The incidence of vasospasm is lower than the incidence of SAH (because only some patients with SAH develop vasospasm). The term “vasospasm” is used in connection with arterial narrowing, which is usually determined by angiography. Clinical vasospasm is very often used synonymously with delayed cerebral ischemia (DCI). This can be specified when used as a vasodilator based on an alternative approach, for example, an increased middle cerebral artery diameter cortical Doppler velocity.

Some degree of angioplasty narrowing will occur in at least two thirds of patients who undergo angiography at 4-12 days after SAH. Approximately ⅓ of patients develop neurological degeneration from these DCIs, although it depends on the patient's diligent monitoring and the efficacy of the preventive measures. Among hospitalized SAH patients, 5 to 10% die from vasoconstriction. Compared with intermediate post-SAH patients, patients with very good condition after SAH develop less vasospasm because they have a small amount of SAH. The presence of a thick, diffuse subarachnoid hemorrhage seen in a computed tomography (CT) scan very close to the bleeding episode is an important diagnostic parameter, and can be used to identify a patient to be treated according to the methods described herein.

The absence of blood in the initial CT scan indicates that vasospasm is highly unlikely to have rebleeding. The risk of vasospasm and consequently the risk of DCI is reduced by factors that reduce the exposure time to coagulation. Conversely, the incidence of vasospasm and DCI is increased by the use of anti-fibrinolytic agents that cause arterial exposures to coagulation and cause as much as possible ischemia by other mechanisms. Poor clinical grading is associated with DCI, presumably because they represent a larger dose of SAH. No clear relationship between age, hypertension, or outcome, DCI, was established. Smokers are susceptible to vasospasm and DCI.

There is evidence that vasospasm can be reduced surgically or pharmacologically, in some cases, by coagulation. There is also data suggesting that DCI can be reduced by hypertension and hypertension (hypercalcemia), and by calcium antagonists. Vasoconstriction may be destroyed by mechanical or pharmacological angioplasty. However, described in embodiments herein is a method for reducing the development of vasospasm and/or preventing the onset of vasospasm.

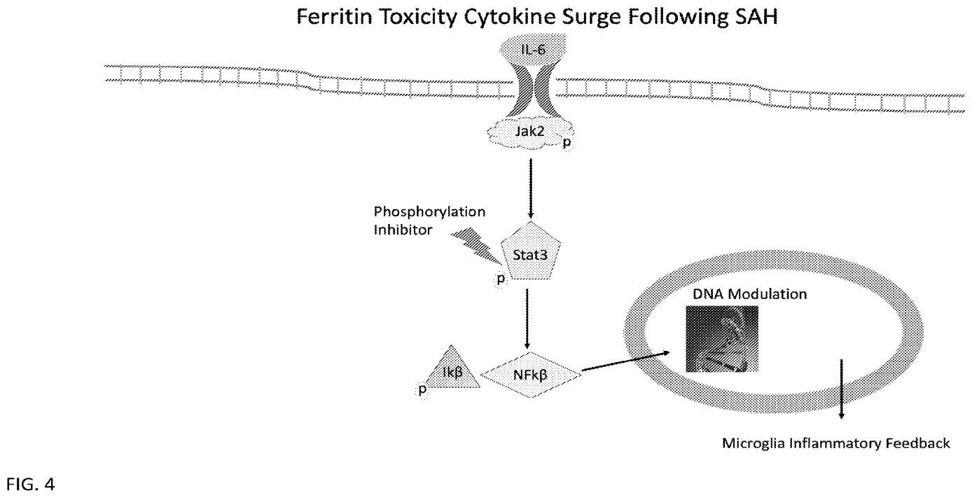

FIG. 4 shows a diagram of the IL-6 cascade that can lead to neuroinflammation. In embodiments herein, there is provided a method for treating subarachnoid hemorrhage (SAH) and/or reducing the incidence of subsequent cerebral vasospasm in a patient, comprising administering a therapeutically effective amount of a first compound or a pharmaceutical composition comprising said first compound to the patient, wherein the first compound comprises a STAT inhibitor compound. In some embodiments, the STAT inhibitor compound comprises a STAT3 inhibitor. In some non-limiting embodiments, the first compound or pharmaceutical composition comprising said first compound is may be administered prior to the vasospasm window (4-21 days following SAH). In other embodiments, the first compound or pharmaceutical composition comprising the first compound is administered immediately following SAH, up to 21 days post-SAH.

### STAT Inhibitors

Many STAT inhibitors target the abnormally activated kinases upstream of STAT. However, direct inhibitors of STAT offer greater selectivity. Thus, there remains a desire for additional STAT inhibitors, especially direct inhibitors of STAT. STAT inhibitors as described herein include inhibitors known in the art. In non-limiting examples described herein the inhibitor may include a peptide which binds at least to STAT3, although it may bind to other STAT proteins.

See, for example, those provided in WO2019/067696 ('696 pub), incorporated herein by reference. In a specific example, the STAT inhibitor is a compound having a structure represented by a formula:

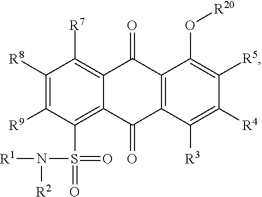

wherein each of R and R2 is independently selected from hydrogen and C1-C6 alkyl; wherein each of R3, R4, R5, R7, R8, and R9 is independently selected from hydrogen, C1-C6 alkyl, C1-C6 alkoxy, halogen, —N02, —NH2, and —OH; and wherein R20 is —C(O)—O—(C1-C6 alkylene), —C(O)—(C1-C6 alkylene), —C(O)—(C1-C6 alkylene)-C(0) OH, —C(0)—NR2 R22, and —(C1-C6 alkylene)-P03H2; wherein each of R2 and R22 is independently selected from hydrogen and C1-C6 alkyl; or a pharmaceutically acceptable salt thereof. Other STAT inhibitors may include WP1066, Resveratrol (SRT501), Stattic, Niclosamide (BAY2353), Stafia-1, STAT3-IN-1, STAT5-IN-1, S31-21, AG490, PIAS protein, cytokine-inducible Src homology 2-containing (CIS) protein, CIS-related protein, suppressor of cytokine signaling-I protein (SOCS-1), tyrphostin, 4,5-dimethoxy-2-nitrobenzoic acid, 4,5-dimehtoxy-2-nitrobenzamide, 4-(phenyl)-amino-6,7-dimethoxyquinazoline, 4-(4′-hydroxyphenyl)-amino-6,7-dimethoxyquinazoline, 4-(3′-bromo-4′-hydroxylphenyl)-amino-6,7-dimethoxyquinalzoline, forskolin, and 3-isobutyl-1-methylxanthine (IBMX). Moreover, Information about STAT inhibitors and methods for their preparation are readily available in the art (see, for example, Kohno M. et al., Progress in Cell Cycle Research, 2003, 5:219-224). In a specific embodiment, the STAT inhibitor is LLL12B (′696 pub; and Chen et al., Sci Rep, 2021, 11(1):6517).

The terms “STAT inhibitor”, “JAK inhibitor”, and “JAK/STAT inhibitor” are used interchangeably herein to refer to any agent capable of down-regulating or otherwise decreasing or suppressing the amount and/or activity of JAK-STAT interactions. JAK inhibitors down-regulate the quantity or activity of JAK molecules. STAT inhibitors down-regulate the quantity or activity of STAT molecules. Inhibition of these cellular components can be achieved by a variety of mechanisms known in the art, including, but not limited to binding directly to JAK (e.g., a JAK-inhibitor compound binding complex, or substrate mimetic), binding directly to STAT, or inhibiting the expression of the gene, which encodes the cellular components. JAK/STAT inhibitors are disclosed in U.S. patent publication 2004/0209799 (Vasios G.).

Examples of JAK/STAT inhibitors which can be useful in the methods of this invention include, but are not limited to: PIAS proteins, which bind and inhibit at the level of the STAT proteins (Chung et al. Science, 1997, 278:1803-1805); cytokine-inducible Src homology 2-containing (CIS) protein, an inhibitor of STAT signaling (Yoshimura et al. EMBO J., 1995, 14:2816-2826); CIS-related proteins, which can inhibit STAT signaling or directly bind to Janus kinases (Yoshimura et al. EMBO J., 1995, 14:2816-2826; Matsumoto et al. Blood, 1997, 89:3148-3154; Starr et al. Nature, 1997, 387:917-921; Endo et al. Nature, 1997, 387:921-924; Naka et al. Nature, 1997, 387:924-929); Suppressor of Cytokine Signaling-I protein (SOCS-1, also referred to as JAB or SSI-1), which appears to associate with all JAKs to block the downstream activation of STAT3 (Ohya et al. J. Biol. Chem., 1997, 272:27178-27182); Tyrphostins, which are derivatives of benzylidene malononitrile, resembling tyrosine and erbstatin moieties (Gazit et al. J. Med. Chem., 1989, 32:2344-2352); AG-490, a member of the tyrophostin family of tyrosine kinase inhibitors (Wang et al. J. Immunol., 1999, 162 (7): 3897-3904, also Kirken et al. J. Leukoc. Biol., 1999, 65:891-899); 4,5-dimethoxy-2-nitrobenzoic acid and 4,5-dimethoxy-2-nitrobenzamide, which specifically inhibit JAK3 (Goodman et al. J. Biol. Chem., 1998, 273:17742-17748); 4-(phenyl)-amino-6,7-dimethoxyquinazoline (parent compound WHI-258) and derivatives of this compound which are structurally-derived from dimethoxyquinazoline compounds (Sudbeck et al. 1999); compounds containing a 4′-OH group, including 4-(4′-hydroxyphenyl)-amino-6,7-dimethoxyquinazoline (WHI-P131), 4-(3′-bromo-4′-hydroxylphenyl)-amino-6,7-dimethoxyquinazoline (WHI-P154), and 4-(3′,5′-dibromo-4′-hydroxylphenyl)-amino-6,7-dimethoxyquinazoline (WHI-P97); WHI-P180, another dimethoxyquinazoline compound (Chen et al. Pharm. Res., 1999, 16 (1): 117-122); and cAMP elevating agents, such as forskolin, a direct activator of adenylate cyclase and dibutyryl cAMP, and 3-isobutyl-1-methylxanthine (IBMX), an inhibitor of cAMP phosphodiesterase (Kolenko et al. Blood, 1999, 93 (7): 2308-2318).

### Dosages, Compositions and Formulations

Depending on the mode of administration, the pharmaceutical composition may comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition. In the treatment conditions which require of inhibition of STAT activity, e.g., STAT3 activity, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

In specific embodiments, the formulation may contain less than about 1 mg/mL of an active ingredient, or between about 1 μg/mL and about 1 mg/ml of the active ingredient, or at least about 1 mg/ml of the active ingredient, or at least about 5 mg/ml of the active ingredient, or at least 100 mg/ml of the active ingredient, or at least about 200 mg/ml of the active ingredient, or at least about 300 mg/ml of the active ingredient. Examples of the active ingredient include an inhibitor of the JAK/STAT pathway, i.e., a STAT inhibitor.

The formulations of the present invention may be for parenteral administration, such as intravenous, intra-arterial, subcutaneous, intradermal, or intramuscular administration (e.g., by injection or by infusion). In some embodiments, the formulation is administered subcutaneously. The formulations can also be delivered transdermally, such as by topically applying the composition to skin (e.g., spreading the composition on skin or loading the composition onto a dermal patch and attaching the dermal patch to the skin).

The formulations of the present disclosure can be administered by infusion or by injection using any suitable device. For example, a formulation of the present invention may be placed into a syringe (e.g., a pre-filled syringe), a pen injection device, an auto-injector device, or a pump device. In some embodiments, the injection device is a multi-dose injector pump device or a multi-dose auto-injector device. The formulation is presented in the device in such a fashion that the formulation is readily able to flow out of the needle upon actuation of an injection device, such as an auto-injector, in order to deliver the peptide drugs. Suitable pen/auto injector devices include, but are not limited to, those pen/auto injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals and the like.

In some embodiments, the formulations of the present invention are provided ready for administration in a vial, a cartridge, or a pre-filled syringe.

In certain embodiments, the formulations include an adjunct stabilizing excipient. The adjunct stabilizing excipient may include, but not be limited to, polypropylene glycol, adducts of polypropylene glycol, and random copolymers comprising propylene oxide units. In embodiments, the polypropylene glycol is a branched polymer, and the branched polymer can be formed by addition of propylene glycol units to a branched or multifunctional alcohol or a branched or multifunctional amine. The branched or multifunctional alcohol can be a sugar, glycerol, pentaerythritol, or triethanolamine. Moreover, the adjunct stabilizing excipient may include, but not be limited to, a hydrophobically modified cellulosic polymer can be selected from the group consisting of a methylcellulose, a hydroxypropyl methylcellulose, a hydroxypropyl cellulose, and a hydroxyethyl cellulose. Furthermore, the adjunct stabilizing excipient may be a polyvinyl alcohol, which can have a molecular weight between about 500 and about 500,000 Daltons, and/or which can have a hydrolysis percent between about 50% and about 100%. In yet other embodiments, the adjunct stabilizing excipient may be a polyoxazoline, which can be selected from the group consisting of poly(2-methyl-2-oxazoline), poly(2-ethyl-2-oxazoline) and poly(2-propyl-2-oxazoline). In embodiments, the polyoxazoline is poly(2-ethyl-2-oxazoline). In embodiments, the polyoxazoline has a weight-average molecular weight between about 1000 and about 500,000 Daltons, or a weight-average molecular weight between about 5000 and about 50,000 Daltons. In certain embodiments, the stabilizing excipient is polyvinylpyrrolidone, which can have a molecular weight between about 1000 and about 1,500,000 Daltons, or a molecular weight between about 5000 and about 200,000 Daltons, or a molecular weight between about 10,000 and about 100,000 Daltons.

In embodiments, the formulation can exclude conventional surfactants. In other embodiments, the formulation further comprises between about 1 and about 5000 ppm of a conventional surfactant, or it comprises between about 1 and about 100 ppm of the conventional surfactant, or it comprises between about 10 and about 5000 ppm of the conventional surfactant, or it comprises between about 100 and 2000 ppm of the conventional surfactant, or it comprises between about 100 and about 2000 ppm of the conventional surfactant. In other embodiments, the formulation further comprises an additional agent selected from the group consisting of preservatives, sugars, polysaccharides, arginine, proline, hyaluronidase, stabilizers, and buffers.

One or more other excipients may be included in the composition of this invention to (1) impart satisfactory processing and compression characteristics to the composition (e.g., adjust the flowability, cohesion and other characteristics of the composition) and (2) give additional desirable physical characteristics to the tables (e.g. color, stability, hardness, disintegration). Mostly the excipients aid in the delayed release of the drug from the composition to achieve regional delivery to the lower GI. As used herein, the term “excipient” may include all excipients present in the dosage form, including all components other than the drug entity and the hydrocolloid gum from higher plants. A plurality of excipient substances may be present in any dosage form, and may include multiple substances having similar pharmaceutical function (e.g., lubricants, binders, diluents) or similar structure (e.g., a mixture of monosaccharides). Preferably the fewer excipients present the better. Such excipients are present in an amount sufficient to provide the composition with the desired delayed release/regional delivery characteristics, hardness rating and handling characteristics and will generally be present at a level of about 2% by weight to about 50% by weight, preferably about 2% by weight to about 40% by weight and more preferably about 2% to about 10% by weight. Excipients may be selected from many categories known in the pharmaceutical arts. The excipients used will be chosen to achieve the desired object of the invention keeping in mind the activity of the drug being used, as well as its physical and chemical characteristics such as water solubility and possible interactions with the excipients to be used.

For example with drugs that are more water soluble, generally a lower percentage by weight of excipients will be used, i.e., less than about 20% or from about 2% to about 15% by weight, preferably no more than about 10% by weight, while for drugs that are less water soluble a higher percentage by weight may be used, e.g., about 20% up to about 40% by weight. These levels may be adjusted to achieve the desired hardness and porosity of the final tablet composition to obtain the delayed release profile.

From the foregoing discussion, it is seen that one aspect of this invention is a particle mass of a solid dosage form that can be administered orally as a tablet. Thus, the composition is neither a liquid nor a gas, but a solid tablet having an amount of drug as a unit dosage. Generally, this unit dosage will be an amount that can be swallowed by a human subject and may vary from a total of about 100 milligrams to about 1500 mg, preferably no more than about 1200 mg and particularly no more than about 800 mg. For children, the size of the tablet may be significantly less than for adults, and for elderly patients who have difficulty swallowing, the total amount may be less than what would be viewed as a normal amount for adults. It is to be understood that the tablets of this invention may be designed as a single tablet having a unit dosage amount or several smaller tablets, e.g., 2-5, may be combined in a capsule for oral administration. The composition used to prepare the tablet may be granulated. In another aspect of the invention, the composition may be formulated to be delivered by a transdermal patch, for example. The patch may further include a penetration enhancer and a pharmaceutically acceptable pressure sensitive adhesive to adhere the patch to a surface of the user. The patch may be embedded with the composition in amounts described herein. The patch may delivery the composition to the user over a predetermined time period to enhance the uptake of the composition. The amount of the pharmaceutical composition present in a drug-containing layer of the patch of the present invention will depend on how soluble it is in the pharmaceutically-acceptable adhesive and excipients present in this layer and how much of the pharmaceutical composition is required in order to achieve the desired therapeutic effect. Typically, the pharmaceutical composition will be present at an amount of 1-10% w/w in the drug-containing layer.

In an exemplary embodiment, the pharmaceutical composition is formulated in accordance with routine procedures as a pharmaceutical composition adapted for oral administration to humans. The amount of the STAT inhibitor which will be effective in the treatment of a particular disease or disorder will depend on the nature of the disease or disorder and can be determined by standard clinical techniques. In addition, in vitro and in vivo assays may optionally be employed to help identify optimal dosage ranges. The dosage will depend on the body weight of the subject. However, in one example suitable dosage ranges for oral administration or parenteral administration may be about 10 μg to 100 mg, 20 μg to 50 mg, 0.1 mg to 20 mg, or 0.5 mg to 10 mg, or any other increments of 0.5 mg within such ranges (calculated either per kg body weight or as total dose per individual). In specific embodiments, the dosage is 1 mg, 2 mg, 3 mg, 4 mg, 5 mg, 6 mg, 7 mg, 8 mg, 9 mg, or 10 mg per kg of body weight. Suitable dosage ranges for intranasal administration are generally about 0.01 μg/kg body weight to 1 mg/kg body weight. Effective doses may be extrapolated from dose-response curves derived from in vitro or animal model test systems. Suppositories generally contain an active ingredient in the range of 0.5% to 10% by weight; oral formulations preferably contain 10% to 95% active ingredient.

Kits are also contemplated as being used in certain aspects of the present invention. For instance, a formulation of the present invention can be included within a kit. A kit can include a container. In one aspect, for instance, the formulation can be comprised within a container that is ready to administer to a subject without having to reconstitute or dilute the formulation. That is, the formulation to be administered can be stored in the container and be readily used as needed. The container can be a device. The device can be a syringe (e.g. pre-filled syringe), a pen injection device, an auto-injector device, a device that can pump or administer the formulation (e.g., automatic or non-automatic external pumps, implantable pumps, etc.) or a perfusion bag. Suitable pen/auto-injector devices include, but are not limited to, those pen/auto-injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals and the like.

### EXAMPLES

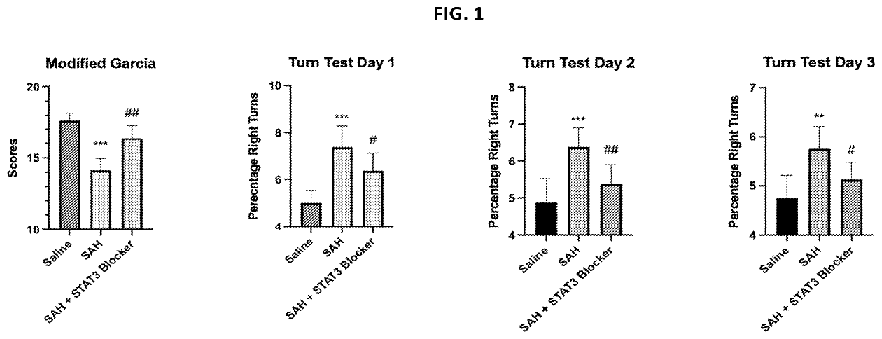

Studies have been conducted herein to determine effectiveness on behavioral data demonstrating both the modified Garcia score and Turn Test as well as reducing vasospasm while using 1) saline, 2) SAH and 3) both SAH and STAT3 Blocker. The data of these studies is provided in FIG. 1.

### Materials and Methods

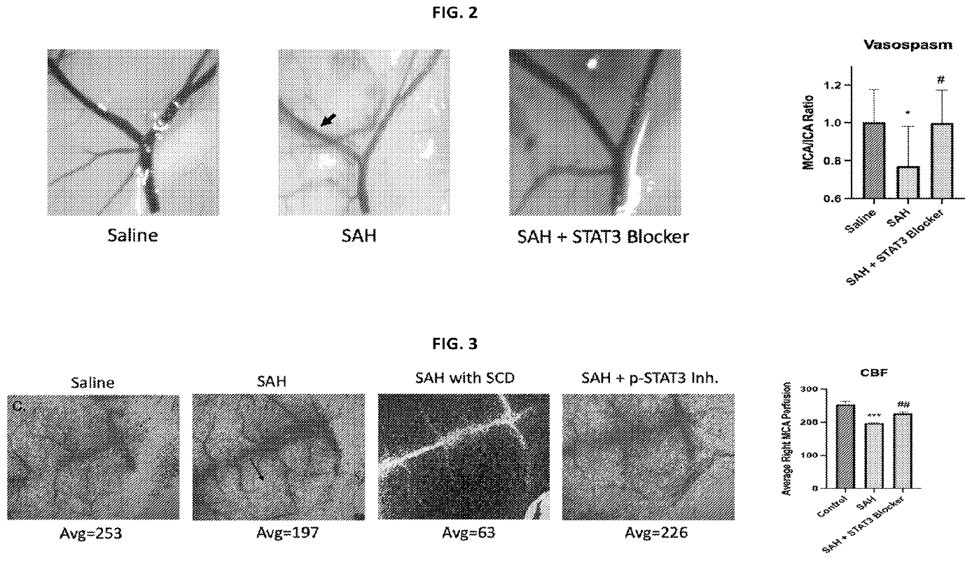

Studies were conducted wherein repetitive dosing on days 1, 2, and 3 following SAH occurred with 5 mg/kg of the p-STAT3 inhibitor. Vasospasm measurements were taken as shown in FIG. 2. P-STAT3 inhibition showed to have protective benefit for cerebral blood flow showing decreased risk for ischemia (see FIG. 3). The immunochemistry data provided in FIG. 3 demonstrates p-STAT3 inhibition reduces systemic neuroinflammation. The STAT inhibitor used in these studies was LLL12B.

### Subarachnoid Hemorrhage

The SAH model utilized modified autologous blood injection to basal cisterns adjacent to right anterior circulation. [Dodd W S, Noda I, Martinez M, Hosaka K, Hoh BL. NLRP3 inhibition attenuates early brain injury and delayed cerebral vasospasm after subarachnoid hemorrhage. J Neuroinflammation. 2021; 18 (1): 163.]. Briefly, mice we anesthetized with 10 mg/kg xylazine and 100 mg/kg ketamine prior to shaving/cleaning the scalp. The tail was prepped, shaved, and cleaned. The mouse's head was fixed to the stereotaxic frame and body temperature maintained at 37° C. using a heating pad. The scalp was incised along the sagittal suture from bregma to the nasal bone. The skin was reflected and held in place by retraction. The burr hole was positioned 5.0 mm rostral to bregma and 0.5 mm right of midline. Next, 150 μL of arterial blood was drawn from the tail artery. 50 μL of blood was drawn up into a Hamilton syringe. The syringe needle was passed at a 30° caudal angle until it reached the skull base. The syringe was left in place and blood was manually injected at a rate of 10 μL/min. The mouse's respiratory rate was closely monitored. Sham procedure was identical to the above except no tail blood was collected but instead saline injected. Once the needle was removed, the skin flap was sutured with 5-0 ethilon suture.

### Vasospasm

Ten mice per group were utilized. Five days post-SAH, mice were deeply anesthetized with ketamine and xylazine. Cardiac catheterization was performed, and perfusion was done with 5 ml PBS followed by 15 ml of 4% paraformaldehyde. 20% India ink was dissolved in 5% gelatin and heated. 2 ml of India ink solution were perfused at a rate of 15 ml/min after the administration of paraformaldehyde. The mouse carcass was then stored at 4° C. for 6 hours to allow hardening. Brains were imaged and the ipsilateral anterior cerebral and middle cerebral artery were captured. The narrowest diameter within the M1 segment was measured as a ratio against average A1 segment diameter. Mice with bifurcated or aplastic vessels were excluded. The ratio is a reliable measure of vasospasm as previously reported [Dodd W S, Noda I, Martinez M, Hosaka K, Hoh B L. NLRP3 inhibition attenuates early brain injury and delayed cerebral vasospasm after subarachnoid hemorrhage. J Neuroinflammation. 2021; 18(1):163]. The results of the experiment are shown in FIG. 2.

### Cerebral Blood Flow

Four mice per group were utilized. Cerebral blood flow was measured with a laser speckle perfusion imager at 48 hours after SAH. The protocol was like that previously described [Cao G, Ye X, Xu Y, Yin M, Chen H, Kou J, Yu B. YiQiFuMai powder injection ameliorates blood-brain barrier dysfunction and brain edema after focal cerebral ischemia-reperfusion injury in mice. Drug Des Devel Ther. 2016; 10:315-25]. Briefly, mice were anesthetized, scalp prepped, and skin removed to reveal skull surface. Sufficient readings were able to be achieved without bone flap removal. The speckle perfusion imager was placed ~10 cm above the skull and leveled to a baseline value of 100 (Pericam PSI System, PeriMed). Recordings were done for 2 minutes per animal. A color-coded image was displayed and converted into numerical scoring based on computer algorithm (PIMsoft, Perimed, Stockholm, Sweden). Average perfusion per animal was documented and an imaging voxel with a calculated mean of 9.7 was used to measure bilateral MCA territory perfusion. A ratio of right to left MCA perfusion was calculated for each animal to allow reliable comparisons between groups and to control for variance in baseline blood pressure differences. See FIG. 3 for results of this experiment.

### Neurologic Evaluation

Eight mice per group were utilized. Mice were observed and scored based on the modified Garcia score and corner test. The modified Garcia score consists of six tests involving assessment of whisker stimulation, climbing, body proprioception, forepaw outstretching, spontaneous activity, and movement of four limbs. [Li R, Ma K, Zhao H, Feng Z, Yang Y, Ge H, Zhang X, Tang J, Yin Y, Liu X, et al. Cattle encephalon glycoside and ignotin reduced white matter injury and prevented post-hemorrhagic hydrocephalus in a rat model of intracerebral hemorrhage. Sci Rep. 2016; 6:35923]. For the corner (turn) test, two 20 cm boards were adjoined at 30° and placed on a clean testing environment. Mice we acclimatized to the apparatus for 10 minutes prior to SAH or saline administration. The mice were tested post SAH as previously described. [Bouet V, Freret T, Toutain J, Divoux D, Boulouard M, Schumann-Bard P. Sensorimotor and cognitive deficits after transient middle cerebral artery occlusion in the mouse. Exp Neurol. 2007; 203(2):555-67.] Briefly, the mouse was placed directly in the middle of the two boards with nose facing the angle. The mouse entered deep into the corner and would rear to turn. The turn was recorded as either right or left. 10 total turns were recorded for each animal per day. The results of these experiments are shown in FIG. 1

### Immunohistochemistry

Six mice per group were utilized. Mice were anesthetized and cardiac perfused with PBS at a rate of 5 ml/min. Brains were removed, placed in plastic cassette with OCT, and submerged in −65° C. isopentane. The blocks were stored at −80° C. On day of sectioning, the OCT blocks were mounted for the Leica CM3050S cryostat (Leica Microsystems) and sliced at a thickness of 14 μm. Slices were collected on a mounted glass slide. The staining protocol was similar to that previously published. [Logsdon A F, Lucke-Wold B P, Turner R C, Li X, Adkins C E, Mohammad A S, Huber J D, Rosen C L, Lockman P R. A mouse model of focal vascular injury induces astrocyte reactivity, tau oligomers, and aberrant behavior. Arch Neurosci. 2017.] After initial serial washes, brain slices were incubated overnight with primary antibodies according to manufacture recommended dilutions. Antibodies used: p-STAT3, Arg1, Cox2 (Cell Signaling, Danvers, MA), iNOS (Abcam, Branford, CT), and TLR-4 (Proteintech, Rosemont, IL). The slides were then rinsed, and Alexa Fluor secondary antibodies were applied to the slides for 3 hours. The slide was rinsed and Vectashield 4′,6-diamidino-2-pheynylindole (DAPI) nuclear counterstain was applied (Vector, Burlingame, CA).

Right frontal and periventricular regions were examined. Ten slides per animal were prepared. Olympus IX71 fluorescent microscope (Olympus America, Center Valley, PA) was used. For fluorescent assays, total corrected cell fluorescent protocol was utilized as previously described. [Logsdon A F, Lucke-Wold B P, Nguyen L, Matsumoto R R, Turner R C, Rosen C L, Huber J D. Salubrinal reduces oxidative stress, neuroinflammation and impulsive-like behavior in a rodent model of traumatic brain injury. Brain Res. 2016; 1643:140-51.]. For cellular morphology assays, total number of cells per high power field were compared as previously described by blinded observer. [Lucke-Wold B P, Logsdon A F, Turner R C, Huber J D, Rosen C L. Endoplasmic reticulum stress modulation as a target for ameliorating effects of blast induced traumatic brain injury. J Neurotrauma. 2017; 34(S1):S62-70.]

While specific embodiments of the subject invention have been disclosed herein, the above specification is illustrative and not restrictive. While this invention has been particularly shown and described with references to preferred embodiments thereof, it will be understood by those skilled in the art that various changes in form and details may be made therein without departing from the scope of the invention encompassed by the appended claims. Many variations of the invention will become apparent to those of skilled art upon review of this specification. Unless otherwise indicated, all numbers expressing reaction conditions, quantities of ingredients, and so forth, as used in this specification and the claims are to be understood as being modified in all instances by the term “about.” Accordingly, unless indicated to the contrary, the numerical parameters set forth herein are approximations that can vary depending upon the desired properties sought to be obtained by the present invention.

What is claimed is:

1. A method for treating subarachnoid hemorrhage (SAH) and/or reducing the incidence of subsequent cerebral vasospasm in a patient, comprising administering a therapeutically effective amount of a first compound or a pharmaceutical composition comprising said first compound to the patient, wherein the first compound comprises a STAT inhibitor compound.

2. The method of claim 1, wherein the STAT inhibitor compound comprises a STAT inhibitor.

3. The method of claim 2, wherein the STAT inhibitor compound comprises a STAT3 inhibitor.

4. The method of claim 1, wherein the patient comprises a patient having SAH.

5. The method of claim 1, wherein the patient comprises a patient exhibiting symptoms of SAH or cerebral vasospasm, or both.

6. The method of claim 1, wherein the administration occurs within the first five (5) days following SAH in the patient.

7. The method of claim 1, wherein the administration occurs at days 1, 2, and 3 following SAH in the patient.

8. The method of claim 1, comprising between 0.1 mg/kg-100 mg/kg of the first compound.

9. The method of claim 7, wherein 5 mg/ml of the first compound is administered on days 1, 2 and 3 following SAH in the patient.

10. A method for preventing cerebral vasospasm in a patient, comprising
identifying the patient is suffering from SAH; and
administering a therapeutically effective amount of a first compound comprising a STAT inhibitor, or a pharmaceutical composition comprising said first compound to the patient.

11. The method of claim 10, wherein the therapeutically effective amount comprises between 0.1 mg/kg-100 mg/kg administered daily for at least 2-5 days following SAH.

12. A method for reducing neural damage caused by SAH, comprising administering a therapeutically effective amount of a first compound or a pharmaceutical composition comprising said first compound to the patient, wherein said first compound inhibits the JAK-STAT pathway.

13. The method of claim 12, wherein the therapeutically effective amount comprises between 0.1 mg/kg-100 mg/kg administered daily for at least 2-5 days following SAH.

14. A method for reducing the severity of cerebral vasospasm in a patient, comprising:
detecting cerebral vasospasm in the patient or identifying the patient is exhibiting symptoms of SAH or cerebral vasospasm; and
administering a therapeutically effective amount of a first compound comprising a STAT3 inhibitor, or a pharmaceutical composition comprising said first compound to the patient.

15. The method of claim 14, wherein the therapeutically effective amount comprises between 0.1 mg/kg-100 mg/kg administered daily for at least 2-5 days following SAH.

16. A method for reducing neural damage caused by SAH in a patient, comprising administering a therapeutically effective amount of a first compound or a pharmaceutical composition comprising said first compound to the patient, wherein said first compound inhibits the JAK-STAT pathway.

17. The method of claim 16, wherein the first compound comprises a STAT inhibitor.

18. The method of claim 16, wherein the STAT inhibitor comprises a STAT3 inhibitor.

19. The method of claim 16, wherein the therapeutically effective amount comprises between 0.1 mg/kg-100 mg/kg administered daily for at least 2-5 days following SAH.

20. The method of any of claims 1-19, wherein the STAT inhibitor is LLL12B.
